(19) United States
(12) Patent Application Publication
(10) Pub. No.: US 2015/0105867 A1
(43) Pub. Date: Apr. 16, 2015

(54) PROSTHETIC SUSPENSION SYSTEM USING INTERLOCKING TEXTURES

(71) Applicant: Jay Novak, Beverly Hills, CA (US)

(72) Inventor: Jay Novak, Beverly Hills, CA (US)

(21) Appl. No.: 14/515,180

(22) Filed: Oct. 15, 2014

Related U.S. Application Data
(60) Provisional application No. 61/891,709, filed on Oct. 16, 2013.

(51) Int. Cl. A61F 2/80; A61F 2/78

(52) U.S. Cl. CPC A61F 2/80; A61F 2/7812
USPC 623/36

(57) ABSTRACT

A prosthetic system includes a gel liner with a patterned texture on its outer surface and a socket with a complementary and interlocking patterned texture on its inner surface. The gel liner is configured to conform and fit to a residual limb, and the socket is configured to fit over the gel liner. A prosthetic system configured in this manner allows for more control of both of vertical slippage and rotational slippage in comparison to the previously known methods of suspension.

### CROSS-REFERENCE TO RELATED PATENT APPLICATIONS

Priority is claimed to U.S. Provisional Patent Application No. 61/891,709 filed Oct. 16, 2013, the disclosure of which is incorporated herein by reference in its entirety.

### FIELD

The field of the present invention is prosthetic devices.

### BACKGROUND

A typical prosthetic device typically includes a gel liner that conforms and adheres to the residual limb and a socket, attached to the prosthetic limb, which fits over the gel liner. The socket itself may include a hard socket molded to the user's residual limb, with the hard socket interfacing with the gel liner. Other systems include both a hard socket and a soft socket, made of a pliable material, with the soft socket permanently lining the hard socket and interfacing with the gel liner.

Careful consideration is typically given to the manner in which the prosthetic device is suspended from the residual limb. Various suspension methods and systems exist, making use of suction, belts, elevated vacuum, and pins, among many other things. The goal of each type of suspension is to create a positive, firm connection between the residual limb and the prosthetic device, making the prosthetic device a natural extension of the residual limb. However, the known methods of suspension suffer from one or both vertical slippage and rotational slippage, causing a weak connection between the prosthetic device and residual limb. Another problem with existing suspension systems is that the wearer may encounter difficulties donning the prosthesis, especially for those in which an elevated vacuum is used.

### SUMMARY

The present invention is directed toward a prosthetic system which provides enhanced resistance to vertical slippage and/or rotational slippage when the prosthesis is fitted to the residual limb of the user.

The prosthetic system includes a gel liner with a patterned texture on its outer surface and a socket with a complementary and interlocking patterned texture on its inner surface. The gel liner is configured to conform and fit to the residual limb, and the socket is configured to fit over the gel liner. Several different options by be incorporated into this basic system to further enhance the resistance to vertical slippage and/or rotational slippage. These different options may be employed individually or in any combination.

As a first option, the socket may include an outer hard socket and an inner soft socket. The inner surface of the soft socket contains the patterned texture, which is complementary to, and interlocks with, the patterned texture of the gel liner.

As a second option, complementary and interlocking patterns may be configured such that affixing the socket to the gel liner requires less energy than removing the socket from the gel liner.

As a third option, the complementary and interlocking patterns of both the gel liner and the socket may be configured such that micro-pockets of vacuum are formed between the respective patterns following fixation of the socket to the gel liner on the residual limb.

As a fourth option, the gel liner and socket may have multiple distinct regions of patterned textures configured to be complementary and interlocking with each other.

Accordingly, an improved prosthetic system is disclosed. Advantages of the improvements will be apparent from the drawings and the description of the preferred embodiment.

### BRIEF DESCRIPTION OF THE DRAWINGS

In the drawings, wherein like reference numerals refer to similar components:

FIG. 1A illustrates a side view of a gel liner.

FIG. 1B illustrates a cross-section view of the interior of a socket.

FIG. 2 illustrates a partial sectional, exploded view of a gel liner with a socket having an outer hard socket and an inner soft socket.

FIG. 3 illustrates an enlarged portion of a gel liner.

FIGS. 4-9 illustrate topographic views of various embodiments of patterned textures.

FIG. 10 illustrates a profile view of one embodiment of a patterned texture.

FIG. 11 illustrates a cross-section view of a gel liner in contact with a socket.

FIG. 12 illustrates a gel liner which includes a pin for further securing the gel liner to a socket.

### DETAILED DESCRIPTION

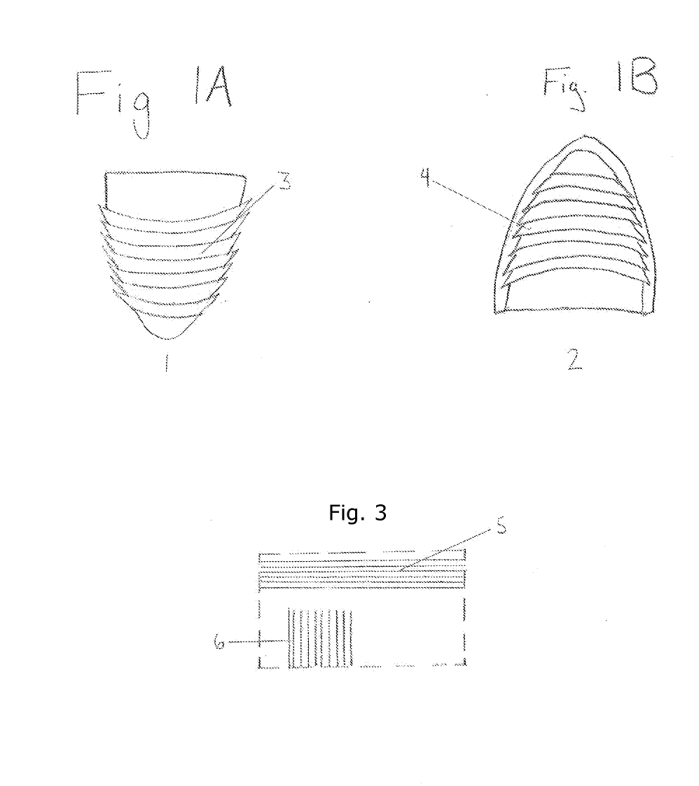

Turning in detail to the drawings, FIG. 1A illustrates a side view of a gel liner 1. Part of the outer surface of the gel liner 1 includes a patterned texture which is used to secure the gel liner to the socket, as shown in FIG. 1B. The complementary and interlocking textures 3 and 4 of the gel liner 1 and the socket 2 are illustrated in FIG. 1B. The textures of each respective part lock the gel liner 1 and the socket 2 together with a bias in the lever purchase toward the negative, i.e. the two parts remain together until a sufficient separation force is applied to remove the socket 2 from the gel liner 1. The bias may also create an impedance to relative rotation between the socket 2 and the gel liner 1. The bias created by the respective complementary textured surfaces means that attaching the socket to the gel liner requires less energy than removing the socket from the gel liner. In one configuration, the complementary and interlocking textures 3 and 4 would have the bias in the lever purchase described above, as well as micro-pockets of vacuum that are created in and between the respective textured surfaces. The gel liner 1 and socket 2 may be made from any appropriate material known in the art.

The gel liner 1 and socket 2 can also comprise two or more discreet regions of complementary and interlocking textures. In this manner, it is possible to better control one or both of the vertical slippage and rotational slippage based caused by the unique circumstances of each wearer's residual limb.

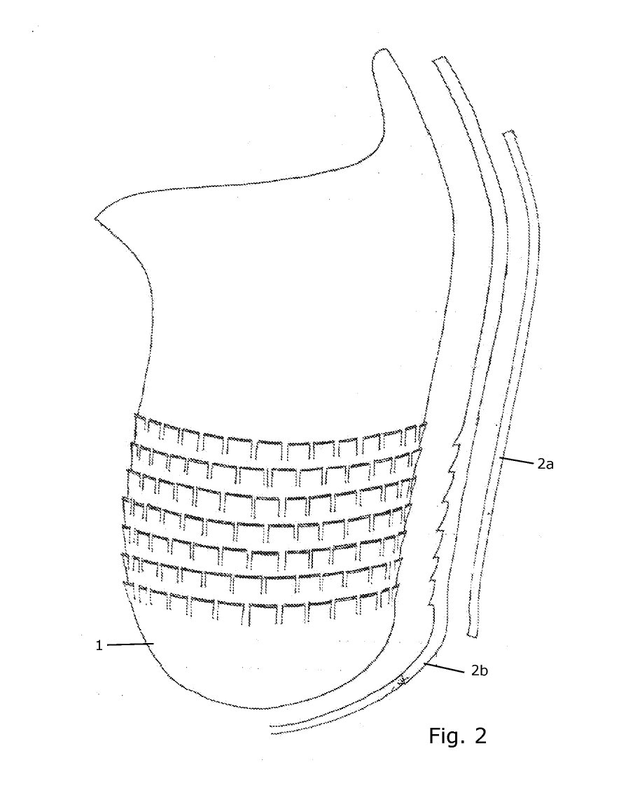

FIG. 2 illustrates a gel liner 1 in combination with a socket 2. The socket 2 includes an outer hard socket 2a and an inner soft socket 2b. During use, the hard socket 2a is secured to the soft socket 2b using techniques that are well-known to those of skill in the art, and the soft socket 2b has an inner surface having interlocking textures, which are configured to be complementary and to interlock with the texture on the outer surface of the gel liner 1. For example, the soli socket 2b may be secured to the hard socket 2a using an adhesive.

FIG. 3 illustrates an enlarged portion of a gel liner. Vertical suspension in this embodiment is achieved through the horizontal ridges 5. Rotational stability in this embodiment is achieved through the vertical ridges 6.

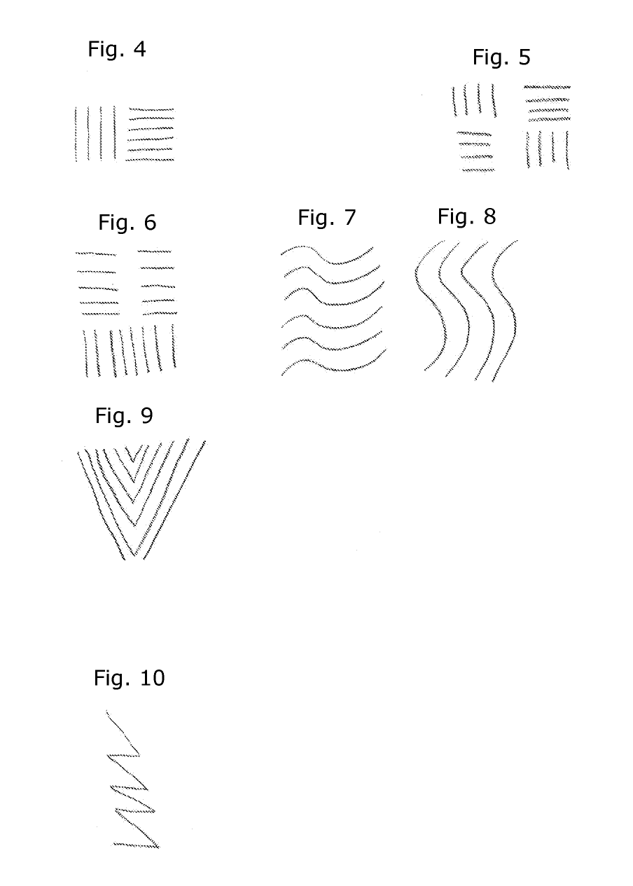

FIGS. 4-9 illustrate topographic views of various embodiments of the patterned textures. The specific angles and depths of the textures are formed to accommodate the unique circumstances of each wearer's residual limb and the needs of the wearer during use of the prosthesis. For example, in the case of a wearer of a prosthesis on a residual arm limb, a wearer who works in an office environment may have completely different needs, as far as resistance to slippage is concerned, than a wearer who works in a manufacturing environment, and different still from a wearer who works in a child care environment. The appropriate configuration of the texture is determined by an examination of several factors, including but not limited to: the body weight of the wearer, the weight of the prosthesis, the type of prosthesis, and the requirements expected of the prosthesis based on activities of the wearer.

As discussed above, the gel liner 1 and socket 2 may comprise two or more discreet regions of complementary and interlocking patterned textures. FIGS. 4-6 show that the patterned textures may be formed from multiple regions of patterned textures configured to be complementary and interlocking with each other. FIGS. 7 and 8 show that the ridges may be formed by non-straight lines as in FIGS. 4-6 and 9, but may also possess more complex, curvilinear topography. Furthermore, a larger patterned texture could be formed from a combination of any of the topographies of FIGS. 4-9, as well as from any other suitable topography which may prove advantageous in creating the desired anti-slippage bias for the prosthesis.

FIG. 10 illustrates a profile view of one embodiment of a patterned texture on one of the gel liner or the socket. This texture is formed from a series of peaks and valleys, the combination of which are formed to be complementary to, and thereby interlock with, a texture on the other of the gel liner or the socket. The complementary patterns need not be exact topological opposites for purposes of interlocking and creating the desired bias. Rather, the complementary patterns are each respectively designed and formed to create the desired anti-slippage bias. For example, the where a series of ridges and valleys are formed as the pattern on the gel liner, with the ridges being formed with well-defined angles, the complementary pattern on the socket may include a series of ridges and valleys, with the ridges being formed by rounded or curved edges.

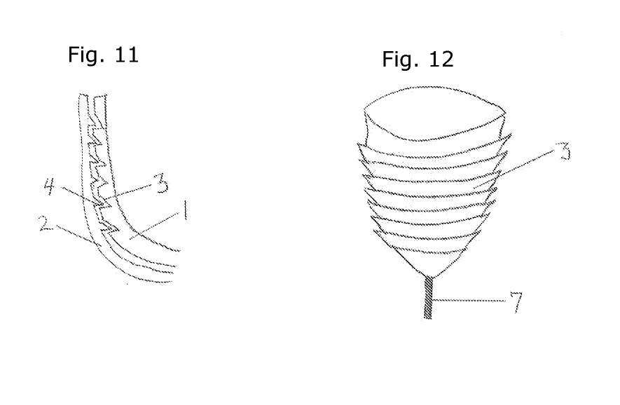

FIG. 11 illustrates a cross-section view of an embodiment of the gel liner in contact with the socket. A system of complementary and interlocking textures 3 and 4 lock the two together with a bias in the lever purchase toward the negative.

FIG. 12 illustrates an embodiment of the gel liner which possesses a pin for further securing the gel liner to the socket. This method of securing a gel liner to a socket is well-known in the art. The gel liner 1 has texture 3 and further comprises pin 7. Pin 7 passes through an opening in socket 2, which has an interior surface with texture 4. In this manner, the socket can be held in contact with the gel liner by way of the complementary and interlocking textures 3 and 4, as well as by way of the pin 7. Furthermore, in one configuration, the complementary and interlocking textures 3 and 4 would have the bias in the lever purchase described above, as well as micro-pockets of vacuum, and the gel liner 1 and socket 2 may further be held in contact by pin 7. Other methods of securing a gel liner to a socket are known to those of skill in the art, and such other methods may also be used in connection with complementary and interlocking textures as described above.

Thus, a prosthetic system is disclosed. While embodiments of this invention have been shown and described, it will be apparent to those skilled in the art that many more modifications are possible without departing from the inventive concepts herein. The invention, therefore, is not to be restricted except in the spirit of the following claims.

What is claimed is:

1. A prosthetic system comprising a gel liner having a first patterned texture on an outer surface and a socket having a second patterned texture on an inner surface, wherein the gel liner is configured to conform and fit to a residual limb and the socket is configured to fit over the gel liner, and wherein the first and second patterned textures are configured to be complementary and interlocking.

2. The prosthetic system of claim 1, wherein the socket comprises an outer hard socket and an inner soft socket, the inner soft socket including the inner surface with the second patterned texture.

3. The prosthetic system of claim 1, wherein the first patterned texture and the second patterned texture are configured such that attaching the socket to the gel liner requires less energy than removing the socket from the gel liner.

4. The prosthetic system of claim 1, wherein the gel liner comprises a pin for securing the gel liner to the socket.

5. The prosthetic system of claim 1, wherein the first and second patterned textures are configured to form micro-pockets of vacuum therebetween when the socket is fit over the gel liner.

6. The prosthetic system of claim 1, wherein the gel liner and the socket each have multiple regions of patterned textures configured to be complementary and interlocking with each other, wherein at least two of the multiple regions of patterned textures on the gel liner have different patterns.

7. The prosthetic system of claim 6, wherein the multiple regions of patterned textures are configured to form micro-pockets of vacuum therebetween when the socket is fit over the gel liner.

8. The prosthetic system of claim 6, wherein the gel liner comprises a pin for securing the gel liner to the socket.

9. A prosthetic system comprising:
a gel liner having a first patterned texture on an outer surface; and
a socket having an outer hard socket and an inner soft socket, with a second patterned texture on an inner soft socket, wherein:
the gel liner is configured to conform and fit to a residual limb and the socket is configured to fit over the gel liner,
the first and second patterned textures are configured to be complementary and interlocking, and
the first patterned texture and the second patterned texture are configured such that attaching the socket to the gel liner requires less energy than removing the socket from the gel liner.

10. The prosthetic system of claim 9, wherein the gel liner comprises a pin for securing the gel liner to the socket.

11. The prosthetic system of claim 9, wherein the first and second patterned textures are configured to loan micro-pockets of vacuum therebetween when the socket is fit over the gel liner.

12. The prosthetic system of claim 9, wherein the gel liner and the socket each have multiple regions of patterned textures configured to be complementary and interlocking with each other, wherein at least two of the multiple regions of patterned textures on the gel liner have different patterns.

13. The prosthetic system of claim 12, wherein the multiple regions of patterned textures are configured to form micro-pockets of vacuum therebetween when the socket is fit over the gel liner.

14. The prosthetic system of claim 12, wherein the gel liner comprises a pin for securing the gel liner to the socket.

15. A prosthetic system comprising:
a gel liner having a first patterned texture cm an crater surface, the first patterned texture having a plurality of first regions; and
a socket having an outer hard socket and an inner soft socket, with a second patterned texture on an inner soft socket, the second patterned texture having a plurality of second regions, wherein:
the gel liner is configured to conform and fit to a residual limb and the socket is configured to fit over the gel liner,
the first and second patterned textures are configured to be complementary and interlocking,
the first patterned texture and the second patterned texture are configured such that attaching the socket to the gel liner requires less energy than removing the socket from the gel liner, and
at least two of the first regions of the first patterned texture have different patterns.

16. The prosthetic system of claim 15, wherein the socket comprises an outer hard socket and an inner soft socket, the inner soft socket including the inner surface with the second patterned texture.

17. The prosthetic system of claim 15, wherein the gel liner comprises a pin for securing the gel liner to the socket.

18. The prosthetic system of claim 15, wherein the first and second patterned textures are configured to form micro-pockets of vacuum therebetween when the socket is fit over the gel liner.
